(19) United States
(12) Patent Application Publication
(10) Pub. No.: US 2008/0010963 A1
(43) Pub. Date: Jan. 17, 2008

(54) Catamenial tampon employing composite yarn as withdrawal cord

(76) Inventors: Jun Takagi, Otsu (JP); Hisao Inuyama, Otsu (JP); Mutsuhiro Ozeki, Ichinomiya (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/891,883

(22) Filed: Aug. 14, 2007

Related U.S. Application Data
(62) Division of application No. 10/624,016, filed on Jul. 21, 2003.
Application No. 10/624,016 is a continuation of application No. PCT/US02/02330, filed on Jan. 24, 2002.
(60) Provisional application No. 60/322,141, filed on Sep. 14, 2001.

(51) Int. Cl. D02G 3/00; A61F 13/20; D02G 3/02

(52) U.S. Cl. 57/12; 57/362; 604/11

(57) ABSTRACT

The present invention is directed to a catamenial tampon which includes a primary absorbent member and a withdrawal cord having a withdrawal portion and an attachment portion. The attachment portion is joined to the primary absorbent member. The withdrawal cord includes a composite yarn, which includes a continuous string, and a secondary absorbent member joined to a part of the continuous string. The continuous string is woven according to a predetermined weaving manner after being provisionally twisted, thereby forming the attachment portion and the withdrawal portion.

### CROSS REFERENCE TO RELATED APPLICATIONS

This application is a divisional of U.S. application Ser. No. 10/624,016 which is a continuation of PCT/US02/02330 filed on Jan. 24, 2002, which claims the benefit of U.S. Provisional Application No. 60/322,141 filed on Sep. 14, 2001, which claims the benefit of PCT/US01/02358 filed on Jan. 25, 2001.

### FIELD OF THE INVENTION

The present invention generally relates to catamenial tampons. More specifically, the present invention relates to catamenial tampons which employ a composite yarn as a withdrawal cord. The present invention also relates to a composite yarn having fleeces intermittently joined thereto and a method for making the same.

### BACKGROUND OF THE INVENTION

A wide variety of absorbent catamenial tampons have long been known in the art. Most currently commercially available tampons include a tampon blank and a withdrawal cord joined to the tampon blank. Such tampons are made by compressing the tampon blank into a substantially cylindrical form. Tampon blanks of a variety of types and constructions have been described in the art. Prior to compression, the blank may be rolled, spirally wound, folded, or assembled as a rectangular pad of absorbent material. Tampons made from a generally rectangular blank of absorbent material have been popular and successful in the market.

The absorbent catamenial tampons now in use typically comprise absorbent tampon blanks which are compressed to a generally cylindrical form about three-eighths to one-half inch (about 1.0 cm to 1.3 cm) in diameter and from about 2 cm to 7 cm in length. These absorbent tampon blanks are usually formed from batts larger in size than the vaginal orifice, which are then compressed to the size (with a corresponding increase in rigidity) indicated above in order to facilitate insertion. As fluid is absorbed, these compressed tampons are expected to re-expand toward their original pre-compressed size, and to eventually become large enough to effectively cover the vaginal cavity against fluid leakage.

Most conventional catamenial tampons are provided with a withdrawal cord which extends out of the user's body following insertion to allow for retrieval and disposal of the used tampon. Typically, this cord does not have any other function associated with it other than the removal of the tampon. The International Publication No. WO 00/61052, published on Oct. 19, 2000, describes a tampon having a secondary absorbent material in addition to a compressed primary absorbent material. The advantages of the secondary absorbent material described in this publication include an ability of the tampon to absorb bypass flow in the early stages of wear, as well as an ability of the tampon to absorb residual fluid which may have been “squeezed out” of a prior tampon during removal. The Publication WO 00/61052 discloses numerous configurations of such a secondary absorbent material including those in which the secondary absorbent material is attached to or is part of a portion of the withdrawal cord, especially that portion of the withdrawal cord which is closest to the primary absorbent member of the tampon.

While the catamenial tampons disclosed in the publication WO 00/61052 effectively absorb bypass flow in the early stages of wear and residual fluid which may have been “squeezed out” of a prior tampon during removal, there is one challenge in providing a tampon such as that described in the publication WO 00/61052 having a secondary absorbent member is to be able to manufacture this member at high speeds and to integrate it efficiently into the remainder of the tampon structure.

Based on the foregoing, there is a need for a catamenial tampon including a withdrawal cord with a secondary absorbent member that can be integrated with the remainder of the tampon easily at high manufacturing speeds. There is also a need for a composite yarn that can be employed in such a catamenial tampon as well as other products, and a need for a method for making such a composite yarn.

### SUMMARY OF THE INVENTION

In one aspect, the present invention is directed to a catamenial tampon, comprising: a primary absorbent member; and a withdrawal cord having a withdrawal portion and an attachment portion. The attachment portion is joined to the primary absorbent member. The withdrawal cord includes a composite yarn which includes a continuous string, and a secondary absorbent member joined to a part of the continuous string. The continuous string which has the secondary absorbent member joined thereto is woven according to a predetermined weaving manner after being provisionally twisted, thereby forming the attachment portion and the withdrawal portion.

In another aspect, the present invention is directed to a composite yarn having thinner portions and thicker portions which are alternatively disposed. The composite yarn comprises: a continuous string; and a plurality of fleeces intermittently joined to the continuous string. The continuous string which has the plurality of fleeces intermittently joined to thereto is woven according to a predetermined weaving manner after being provisionally twisted, thereby forming the thinner portions and thicker portions.

In yet another aspect, the present invention is directed to a method of making a composite yarn having thinner portions and thicker portions which are alternatively disposed. The method comprises the steps of: supplying a continuous string; intermittently joining a plurality of fleeces to the continuous string; provisionally twisting the continuous string which has the plurality of fleeces intermittently joined to thereto; and weaving the twisted continuous string according to a predetermined weaving manner.

The foregoing answers the need for a catamenial tampon including a withdrawal cord with a secondary absorbent member that can be integrated with the remainder of the tampon easily at high manufacturing speeds. The foregoing also answers the need for a composite yarn that can be employed in such a catamenial tampon as well as other products, and a need for a method for making such a composite yarn.

These and other features, aspects, and advantages of the present invention will become evident to those skilled in the art from reading of the present disclosure.

### BRIEF DESCRIPTION OF THE DRAWINGS

While the specification concludes with claims particularly pointing out and distinctly claiming the invention, it is believed that the invention will be better understood from the following description of preferred embodiments taken in conjunction with the accompanying drawings wherein like designations are used to designate substantially identical elements, and in which:

FIG. 1 is a front view of a catamenial tampon which is one preferred embodiment of the present invention;

FIG. 2 is a perspective view of a typical tampon blank prior compression into the tampon shown in FIG. 1;

FIG. 3 is a schematic diagram of a composite yarn which will be used for the withdrawal cord of the tampon shown in FIG. 1;

FIG. 4 is a schematic diagram of a preferred apparatus for making a preferred composite yarn;

FIG. 5 is a schematic diagram of yarn loops which are woven with a tubular weaver having one needle;

FIG. 6 is a schematic diagram of yarn loops which are woven with a tubular weaver having two needles;

FIG. 7 is a schematic diagram of one preferred example of a resultant composite yarn produced from the apparatus shown in FIG. 4;

FIG. 8 is a schematic diagram of one preferred embodiment of the front rollers shown in FIG. 4 which are supplied with two continuous strings; and

FIG. 9 is a schematic diagram of another preferred embodiment of the front rollers shown in FIG. 4 which are supplied with three continuous strings.

### DETAILED DESCRIPTION OF THE INVENTION

All cited references are incorporated herein by reference in their entirety. Citation of any reference is not an admission regarding any determination as to its availability as prior art to the claimed invention.

Herein, the terms “comprise”, “include” and “contain” mean that other element(s) and step(s) which do not affect the end result can be added. These terms encompass the terms “consisting of” and “consisting essentially of”.

Herein, the terms “joined” and “joining” encompass configurations whereby an element is directly secured to another by affixing the element directly to the other element, and configurations whereby the element is indirectly secured to the other element by affixing the element to intermediate member(s) which in turn are affixed to the other element.

Herein, the term “tampon” refers to any type of absorbent structure which is inserted into the vaginal canal or other body cavities for the absorption of fluid therefrom. Typically, tampons are constructed from an absorbent material which has been compressed in either the radial direction, the axial direction, or both in order to provide a tampon which is of a size and stability to allow insertion within the vagina or other body cavity. A tampon which has been so compressed is referred to herein as a “self-sustaining” form. That is, the degree of compression applied to the absorbent material of the tampon blank is sufficient so that in the subsequent absence of the external forces, the resultant tampon will tend to retain its general shape and size.

It will be understood by one of skill in the art that this self-sustaining form need not, and preferably does not persist during actual use of the tampon. That is once the tampon is inserted and begins to acquire fluid, the tampon will begin to expand and can lose its self-sustaining form.

Herein, the terms “blank” or “tampon blank” are intended to be interchangeable and refer to a construction of absorbent material prior to the compression of such construction into a tampon as described above. Tampon blanks are sometimes referred to as a softwind, and the term “blank” is intended to include such a term as well.

Herein, the terms “vaginal cavity,” “within the vagina” and “vaginal interior,” are intended to be synonymous and refer to the internal genitalia of the human female in the pudendal region of the body. Herein, the term “vaginal cavity” is intended to refer to the space located between the introitus of the vagina (sometimes referred to as the sphincter of the vagina) and the cervix and is not intended to include the interlabial space, including the floor of vestibule. The externally visible genitalia generally is not included within the term “vaginal cavity”.

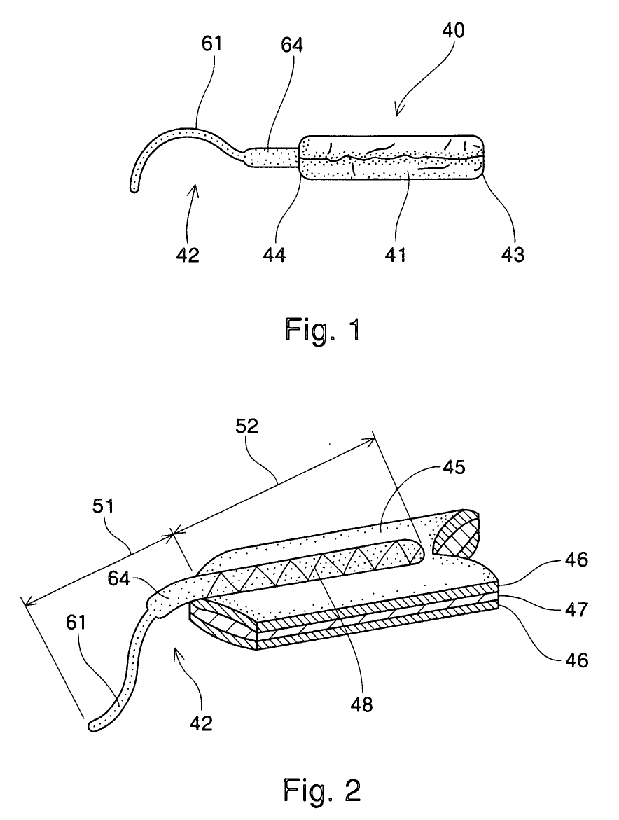

FIG. 1 is a front view of a catamenial tampon 40 which is one preferred embodiment of the present invention. Referring to FIG. 1, the tampon 40 includes a primary absorbent member (sometimes also referred to as the “absorbent core”) 41 and a withdrawal cord 42 which is joined to the primary absorbent member 41. The primary absorbent member 41 of the tampon 40 has an insertion end 43 and a withdrawal end 44.

The withdrawal cord 42 includes (or is formed by) a composite yarn which includes a continuous string 61 and a secondary absorbent member 64 joined to a part of the continuous string 61. The primary absorbent member 41 can be compressed into a generally cylindrical configuration in the radial direction, the axial direction, or in both the radial and axial directions. While the primary absorbent member 41 is preferably compressed into a substantially cylindrical configuration as shown in FIG. 1, other shapes are also possible. These can include shapes having a cross section which can be described as rectangular, triangular, trapezoidal, semi-circular, or other suitable shapes.

The primary absorbent member 41 of the tampon 40 can be formed from any suitable tampon blank. A preferred example of the primary absorbent member 41 is a tampon blank 45 which is shown in FIG. 2. The withdrawal cord 42 has a withdrawal portion 51 which is used for the withdrawal of the tampon 40 after use, and an attachment portion 52 which is used for the attachment of the cord 42 to the primary absorbent member 41 (or the tampon blank 45). The withdrawal portion 51 includes the continuous string 61 and the secondary absorbent member 64 joined to the continuous string 61. The secondary absorbent member 64 and the continuous string 61 extend into the tampon blank 45 to form the attachment portion 52. The withdrawal cord 42 is joined to the tampon blank 45 through the attachment portion 52.

In a preferred embodiment, the withdrawal cord 42 is joined to the tampon blank 45 prior to the compression of the blank 45 to a self-sustaining form (i.e., the formation of the absorbent member 41). Alternatively, the withdrawal cord 42 can be joined to the tampon blank 45 simultaneously with the compression of the blank 45. In either of the above mentioned manners, the withdrawal cord 42 is preferably not compressed; or, if compressed, is not compressed to the same degree as the primary absorbent member 41.

The tampon blank 45 which will be compressed to form the primary absorbent member 41 can have any suitable shape, size, material, and structure. In the embodiment shown in FIG. 2, the blank 45 is a batt of absorbent material which is in a chevron shape.

While the tampon blank 45 shown in FIG. 2 generally has a chevron shape, other shapes such as rectangular, trapezoidal, triangular, and hemispherical shapes are also acceptable. The blank 45 can be formed by a unitary member structure or a laminate structure which includes discrete layers. When the blank 45 has such a laminate structure, the layers can include different materials (or same materials if desired). For example, one layer includes primarily rayon, while another layer (or layers) includes primarily cotton.

In the embodiment shown in FIG. 2, the blank 45 comprises outer layers 46 and at least one intermediate layer 47 positioned between the outer layers 46. Alternatively, the blank 45 can comprise a folded structure, can be rolled, can comprise a “petal” structure or any other of the structures which are known in the art with respect to tampon blanks.

The tampon blank 45 can be constructed from a wide variety of liquid-absorbing materials commonly used in absorbent articles such as rayon, cotton, or comminuted wood pulp which is generally referred to as airfelt. Examples of other suitable absorbent materials include creped cellulose wadding; meltblown polymers including coform; chemically stiffened, modified or cross-linked cellulosic fibers; synthetic fibers such as crimped polyester fibers; peat moss; foam; tissue including tissue wraps and tissue laminates; or any equivalent material or combinations of materials, or mixtures of these. Preferred absorbent materials comprise cotton, rayon (including tri-lobal and conventional rayon fibers, and needle punched rayon), folded tissues, and woven or nonwoven materials of synthetic and/or natural fibers. The tampon blank 45 can include a single or combination of such materials. For example, the blank 45 can include a uniform material of a unitary material of rayon or cotton, or a blended material of rayon and cotton. Additionally, superabsorbent materials, such as superabsorbent polymers or absorbent gelling materials can be incorporated into the tampon 40.

Preferably, the tampon blank 45 is formed of a soft absorbent material such as rayon, cotton (including either long fiber cotton or cotton linters) or other suitable natural or synthetic fibers or sheeting. The materials for the blank 45 can be either a fabric, web, or batt that is formed by any suitable process known in the art such as airlaying, carding, wetlaying, hydroentangling, or other known techniques. The rayon material used in the tampon blank 45 can be any suitable type typically used in disposable absorbent articles known in the art. Any suitable cotton material can also be used in the tampon blank 45. Suitable cotton material includes, long fiber cotton, short fiber cotton, cotton linters, T-fiber cotton, card strips, and comber cotton. Preferably, such cotton materials should be a scoured & bleached cotton absorbent with a glycerin finish, a leomin finish, or other suitable finish.

In the preferred embodiment shown in FIG. 2, the outer layer 46 is a batt formed by a rayon material which is available from Acordis Fibers Ltd. as Galaxy rayon, while the intermediate layer 47 is a batt formed by a cotton material which is available from Acordis Fibers Ltd.

The absorbent material of the tampon blank 45 can be surrounded with a liquid permeable overlap material, if desired. Suitable overlap materials can comprise rayon, cotton, bicomponent fibers, or other natural or synthetic fibers known in the art.

The tampon blank 45 can be any suitable size and thickness suitable for compression into a tampon having a size which facilitates insertion. A size similar to those of conventional currently available tampons has been found to work well. A typical size for such blanks can be about 9 cm in length and about 4.5 cm in width. One preferred range for the overall basis weight is from about 150 g/m2 to about 750 g/m2. Optionally, blanks 28 which are shorter and wider than the ranges given above can also be desirable in order to facilitate width-wise expansion of the tampon in use.

The withdrawal cord 42 is for removal of the tampon 40 after use. The withdrawal cord 42 is joined to at least the primary absorbent member 41 and extends beyond the withdrawal end 44. The withdrawal cord 42 can be attached in any suitable manner known in the art including a stitching attachment, an adhesive attachment, or a combination thereof. Preferably, the withdrawal cord 42 is attached to the primary absorbent member 41 by stitching the attachment portion 52 onto the tampon blank 45 (i.e., the primary absorbent member 41) as shown in FIG. 2. Any stitching manner known in the art can be used. In a preferred embodiment, the withdrawal cord 42 is stitched onto the tampon blank 45 according to the stitching manner called “Double Ring Stitching” which is described in the Japanese Industrial Standards (JIS) No. B 9070.

The whole part of the secondary absorbent member 64 of the withdrawal cord 42 may be stitched onto the tampon blank 45 by a thread 48. Preferably, as shown in FIG. 2, one part of the secondary absorbent member 64 of the withdrawal cord 42 is stitched onto the tampon blank 45 by the thread 48, while the other part (i.e., the un-stitched part, which has a length of, for example, about 1 inch (2.54 cm)) of the secondary absorbent member 64 goes outside the tampon blank 45 for a better absorption for fluids which may be squeezed out of the tampon blank 45 or the primary absorbent member 41. If desired, the whole of the withdrawal portion 51 of the withdrawal cord 42 may be stitched in addition to the attachment portion 52. Any suitable thread known in the art can be used for stitching.

The attachment portion 52 of the withdrawal cord 42 can be joined to any suitable location on the tampon 40. In the embodiment shown in FIG. 2, the attachment portion 52 of the withdrawal cord 42 is joined to the tampon blank 45 so that the withdrawal portion 51 extends freely beyond the withdrawal end 44. Preferably, the withdrawal cord 42 is flexible.

The withdrawal cord 42 is preferably absorbent at the location of the attachment to the primary absorbent member 41 (i.e., at the attachment portion 52). The secondary absorbent member 64 is also preferably absorbent, however, the rest of the withdrawal cord 42 in the withdrawal portion 51 is preferably non-absorbent. Herein, the term “non-absorbent” refers to a structure that does not retain a significant portion of deposited fluid in its structure. The entire withdrawal cord 42 can be made non-absorbent, if desired. The materials comprising the withdrawal cord can be inherently non-wettable or hydrophobic, or they can be treated to provide such properties. For example, a coating of wax can be applied to the withdrawal cord 42 to decrease or eliminate its absorbency. The withdrawal cord 42 does not necessarily need to be non-wicking, even if a non-absorbent withdrawal cord is desired. For example, it can be desirable to provide a withdrawal portion 51 of the withdrawal cord 42 in which at least a portion of the cord 42 has a tendency to wick deposited fluid upwardly toward the primary absorbent member 41 and into the structure thereof.

The withdrawal cord 42 does not need to have uniform properties throughout its length. For example, the attachment portion 52 of the withdrawal cord 42 can be absorbent while the withdrawal portion 51 can be non-absorbent. Other properties such as wicking ability, hydrophilicity, density, capillary size, width, thickness, and the like can also vary along the length of the withdrawal cord 42.

Preferably, the withdrawal cord 42 is provided with a wicking mechanism to preferentially direct or wick acquired fluid toward the body of the primary absorbent member 41. One example of such a driving force is produced by a hydrophilicity gradient. Other examples of the wicking mechanisms include a density gradient, a capillary gradient, and an osmotic driving force. Capillary channel fibers can optionally be incorporated into the withdrawal cord 42 in order to provide the driving force for acquired fluid described herein. A preferred wicking mechanism which preferentially directs acquired fluid toward the body of the primary absorbent member 41 is disclosed in the International Publication No. WO 00/61052.

Preferably, the density of material which comprises the withdrawal cord 42 is lower than the density of the primary absorbent member 41. The withdrawal cord 42 is preferably not compressed during formation of the tampon 40.

To form a tampon ready for use, the tampon blank 45 is typically compressed and heat conditioned in any suitable conventional manner. Pressures and temperatures suitable for this purpose are well known in the art. Typically, the blank 45 is compressed in both the radial and axial direction using any means well known in the art.

FIG. 3 is a schematic diagram of a composite yarn 60 which will be preferably used in the tampon 40 shown in FIG. 1, i.e., the composite yarn 60 is used as the withdrawal cord 42 of the tampon 40 after being cut at an appropriate length. The composite yarn 60 is described in detail hereinafter as well as its manufacturing processes.

Referring to FIG. 3, the composite yarn 60 has thinner portions I and thicker portions 2 which are provided alternatively and intermittently along the lengthwise axis LA of the composite yarn 60. The thicker portion 2 will be used as the secondary absorbent member 64 of the withdrawal cord 42 in a catamenial tampon. The composite yarn 60 comprises a continuous string 61 which forms the thinner portions 1, and fleeces 62 which are joined intermittently to the continuous string 61 to form the thicker portions 2. The fleeces 62 are the materials for the secondary absorbent member 64.

Herein, the term “continuous string” refers to a string which continuously passes through a composite yarn along the lengthwise axis in the whole length of the composite yarn. The continuous string may include a single continuous string, or if desired, a plurality of continuous strings.

Herein, the term “thinner portion” refers to a portion of a composite yarn which has a smaller diameter than other portions.

Herein, the term “thicker portion” refers to a portion of a composite yarn which has a greater diameter than other portions.

The length LG1 of the thinner portions 1 and the length LG2 of the thicker portions 2 are properly selected so that those can have appropriate lengths for the withdrawal portions 51 and the attachment portions 52 of the withdrawal cords 42 by cutting. Preferably, the length LG1 of the thinner portion 1 is from about 70 mm to about 200 mm, and the length LG2 of the thicker portion 2 is from about 30 mm to about 70 mm. These lengths LG1 and LG2 are determined depending on the tampon design including the sizes and the expected absorbency range of the tampon.

The diameter ratio of the thinner portion 1 to the thicker portion 2 is at least about 1.5, and preferably from about 2 to about 4. In a preferred embodiment, the diameter ratio of the thinner portion 1 to the thicker portion 2 is in the rage of from about 2.5 to about 3.

The continuous string 61 may be formed by any material and string formation method known in the art. Suitable materials include synthetic fibers such as nylon and polyester; natural fibers such as cotton; and regenerated fibers such as rayon. In a preferred embodiment, the continuous string 61 is made of cotton. A preferred continuous string material includes 6 or 7 plies of bleached cotton which is finished with a repellent material such as a wax.

The fleeces 62 (i.e., the materials for the secondary absorbent member 64) may be formed by drafting a sliver of any material known in the art. Suitable materials for such a sliver include spun yarns or filaments of synthetic fibers such as nylon and polyester; spun yarns of cotton or other natural fibers; and spun yarns of regenerated fibers such as rayon. The same material which is used for the tampon blank 45 may be used for the fleeces 62, if desired.

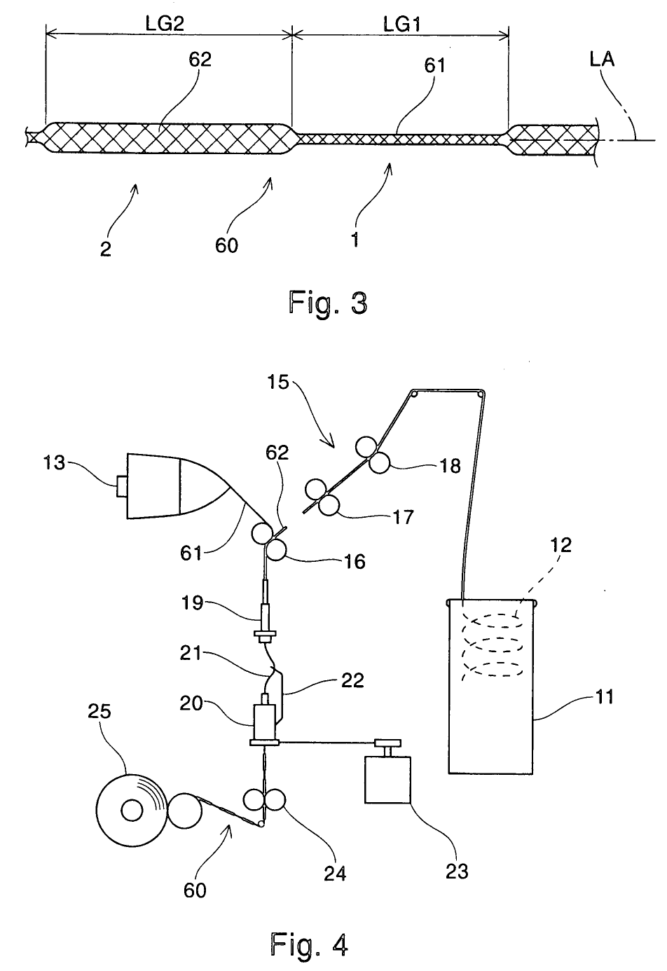

A preferred apparatus for making a preferred composite yarn is described hereinafter. Referring to FIG. 4, a can 11 contains a sliver 12. A bobbin 13 contains a spun yarn which is wound therein and will constitute the continuous string 61. A three-lines drafting machine 15 includes front rollers 16, middle rollers 17, and back rollers 18. The middle rollers 17 and back rollers 18 are designed to operate in tandem at a predetermined draft ratio. A preliminary twist hollow spindle 19 is situated below the front rollers 16. A tubular weaver 20 includes a needle 21 and a guide bar 22 which is driven by a motor 23. A preferred tubular weaver is disclosed in Japanese Patent Laid-Open (Kokai) Publication No. H2000-212865. A resultant composite yarn 25 which has thinner portions and thicker portions will be wound after take-up rollers 24.

In operation, the sliver 12 which is supplied from the can 11 is drawn at a predetermined draft ratio between the back rollers 18 and the middle rollers 17. The sliver 12 is then drawn by the front rollers 16 which rotate at a higher speed than the middle rollers 17. Since the middle rollers 17 and the back rollers 18 stop their rotations simultaneously at a given cycle, a fleece which is between the front rollers 16 and the middle rollers 17 is cut in the given cycle, whereby fleeces 62 having a given length are intermittently supplied to the front rollers 16. The continuous string 61 which is wound on the bobbin 13 is also supplied to the front rollers 16. The intermittently supplied fleeces having a given length is then joined to the continuous string 61 intermittently by the front rollers 16.

The continuous string 61 having the fleeces 62 intermittently joined thereto (hereinafter referred to as “intermediate member”) is then preliminarily twisted in order to increase the strength of the resultant composite yarn 60. The preliminary twist may be performed, for example, with a V-shaped guide (not shown in FIG. 4) which is attached at the outlet of the hollow spindle 19, by looping the continuous string 61 having the fleeces 62 intermittently joined thereto once around the V-shaped guide.

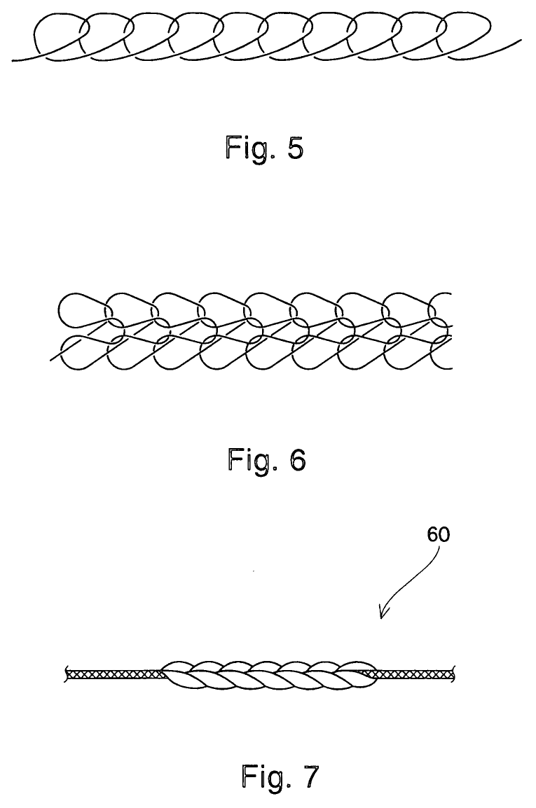

After the preliminary twist spindle 19 applies a preliminary twist to the intermediate member (i.e., the continuous string 61 having the fleeces joined thereto), the twisted intermediate member is then promptly woven in a predetermined weaving manner. Preferred predetermined weaving manners include a traversal weaving manner, a horizontal weaving manner and a tubular weaving manner. In a preferred embodiment, the predetermined weaving manner is a tubular weaving manner, which is performed by using a tubular weaver having from one to four needles, for example. FIG. 5 is a schematic diagram of yarn loops which are woven with a tubular weaver having one needle. FIG. 6 is a schematic diagram of yarn loops which are woven with a tubular weaver having two needles. In a preferred embodiment, the twisted intermediate member is woven according to the loop structure depicted in FIG. 5. The resultant woven member (i.e., a composite yarn 60) is then taken up by the take-up roller 24, and wound therein. FIG. 7 is a schematic diagram of one example of the resultant composite yarn 60.

An apparatus which is preferably used to produce a preferred composite yarn is available from Ozeki Techno K.K. (Aichi, Japan) under Code No. ON-1001 KNIT SPIN.

The apparatus shown in FIG. 4 is used for making a composite yarn which employs a single continuous string 61. In alternative preferred embodiments, the apparatus shown in FIG. 4 can be modified for making a composite yarn which employs a plurality of continuous strings. In such embodiments, an additional bobbin(s) which contains an additional spun yarn is provided (not shown in Figs.) Such an additional bobbin(s), together with the bobbin 13, will supply the plurality of continuous strings to the front rollers 16 in parallel.

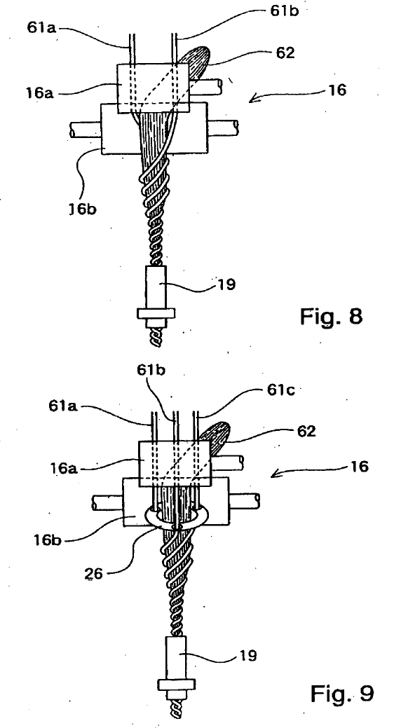

In one preferred embodiment, two continuous strings 61a and 61b are supplied to the front rollers 16 in parallel as shown in FIG. 8. Referring to FIG. 8, the front rollers 16 include an upper roller 16a and a lower roller 16b. The two continuous strings 61a and 61b which are kept at a predetermined distance (e.g., about 4 mm) by a guide (not shown in Figs.) are supplied to the front rollers 16 together with the fleeces 62 which are intermittently supplied such that the two continuous strings 61a and 61b sandwich the fleeces 62 as shown in FIG. 8. After the front rollers 16 join the fleeces 62 to the two continuous strings 61a and 61b intermittently, the intermediate member is preliminarily twisted by the preliminary twist spindle 19 such that the two continuous strings 61a and 61b hold the fleeces 62 in a spiral manner as shown in FIG. 8.

In an alternative preferred embodiment, three continuous strings 61a, 61b and 61c are supplied to the front rollers 16 in parallel as shown in FIG. 9. Referring to FIG. 9, the front rollers 16 include an upper roller 16a and a lower roller 16b. The three continuous strings 61a, 61b and 61c which are kept at a predetermined distance each other (e.g., about 4 mm) by a guide 26 are supplied to the front rollers 16 together with the fleeces 62 which are intermittently supplied such that the three continuous strings 61a, 61b and 61c surround the fleeces 62 as shown in FIG. 9. After the front rollers 16 join the fleeces 62 to the three continuous strings 61a, 61b and 61c intermittently, the intermediate member is preliminarily twisted by the preliminary twist spindle 19 such that the three continuous strings 61a, 61b and 61c hold the fleeces 62 in a spiral manner as shown in FIG. 9.

By employing a plurality of continuous strings in a composite yarn, it is possible to reduce occurrence of fuzz (or fluff) which may be produced in the resultant composite yarn. In addition, by increasing the number (i.e., two, three, or more) of continuous strings in a composite yarn, it is possible to produce a more symmetric composite yarn, and also to reduce potential dispersion in the pitch and length of the thicker portions of the resultant composite yarn.

The composite yarn 60 produced from the above manufacturing processes is cut at an appropriate interval to produce withdrawal cords 42 which will be used in tampons 40 of the present invention. The thinner portion 1 is used as the withdrawal portion 51 of the withdrawal cords 42, while the thicker portion 2 is used as the attachment portion 52. In a preferred embodiment, the composite yarn 60 is cut right after the thicker portion 2 of the yarn 60 (which will become the attachment portion 52 of the withdrawal cords 42) is joined to the primary absorbent member 41.

By using the composite yarn described above as a withdrawal cord, a catamenial tampon which includes a secondary absorbent member can be manufactured quickly and inexpensively. In addition, the withdrawal cord can be integrated with the remainder of the tampon easily at high manufacturing speeds.

It is understood that the examples and embodiments described herein are for illustrative purpose only and that various modifications or changes will be suggested to one skilled in the art without departing from the scope of the present invention.

What is claimed is:

1. A method of making a composite yarn having thinner portions and thicker portions which are alternatively disposed, comprising the steps of:
supplying a continuous string;
intermittently joining a plurality of fleeces to the continuous string;
provisionally twisting the continuous string which has the plurality of fleeces intermittently joined to thereto; and
weaving the twisted continuous string according to a predetermined weaving manner.

2. The method of claim 1, wherein the continuous string includes a plurality of strings.

3. The method of claim 1, wherein the step of intermittently joining a plurality of fleeces to the continuous string includes a step of guiding the plurality of strings such that the fleeces are sandwiched or surrounded by the strings.
